(19) United States
(12) Patent Application Publication
(10) Pub. No.: US 2012/0172762 A1
(43) Pub. Date: Jul. 5, 2012

(54) DEVICE AND METHOD OF AUTOMATIC CALIBRATION OF A TENSOR IN ARTHROPLASTY PROCEDURES

(75) Inventors: Anthony Boyer, Echirolles (FR); François Gougeon, Roubaix (FR); Stéphane Lavallee, St Martin D'Uriage (FR)

(73) Assignee: BLUE ORTHO, La Tronche (FR)

(21) Appl. No.: 13/319,616

(22) PCT Filed: May 11, 2010
(86) PCT No.: PCT/IB2010/001807

§ 371 Date: Mar. 19, 2012

Related U.S. Application Data
(60) Provisional application No. 61/178,141, filed on May 14, 2009.

(51) Int. Cl. A61B 5/103

(52) U.S. Cl. 600/587

(57) ABSTRACT

The present invention relates to a computer assisted surgical navigation method for determining the patient-specific pressure that must be applied on one or both sides of a joint during the ligament balancing steps of an arthroplasty procedure, said method comprising:
inserting a tensor (22) inside the joint between a first bone (8) and a second bone (2) when the joint is in a given angular orientation;
controlling the pressure applied by the tensor (22) to the joint so as to make it increase from a minimum value to a maximum value;
measuring the evolution of the distance between the first and the second bones during said pressure increase;
determining the relationship between said distance and said pressure;
processing said relationship to determine said patient-specific pressure to apply to the joint.

### PURPOSE OF THE INVENTION

The present invention relates to a method and a device that determines the Patient-Specific Pressure (PSP) that needs to be applied between two bones during surgical joint reconstruction (arthroplasty) in order to determine an optimal balancing of the joint.

### BACKGROUND OF THE INVENTION

It is known that some navigation systems are tracking Instrument position during their position adjustment. The tracking technology of trackers and navigation system is independent of the invention, provided that the trackers are tracked in real-time by the navigation system. It includes, but is not limited to optical active technology with active infrared Light Emitting Diodes (LEDs) on trackers, optical passive technology with passive retro-reflective markers on trackers, or magnetic technology, radio-frequency technology, ultrasonic technology, gyroscopic devices and accelerometers, mechanical arms with encoders. Those tracking technologies are known as prior art.

It is known that tensors are used during arthroplasty procedures—such as knee, hip or shoulder arthroplasty—in order to control the ligament tensions. There are multiple tensor designs which use various technologies to generate a pressure between the femur and the tibia, such as hydraulic devices, pneumatic devices, mechanical systems with actuators, springs, or simply manual mechanical systems activated by the human operator. It is also known that several sensors can be attached to tensors in order to measure the pressures or forces applied between the bones.

It is also known that some sensors can be used to measure the distance between the two bones during the use of a tensor, on both medial and lateral parts. In a first group of solutions, a standard distance sensor is used to measure the gap between two plates of the tensor, one plate that pushes the femur and one plate that pushes the tibia (in the case of knee arthroplasty). Such sensor can be also the result of the actuation mechanism, for instance if a hydraulic sensor is used, the volume of liquid inserted between the two plates gives directly an estimate of the distance between the two plates. A second group of solutions use a navigation system to measure the distance between the two bones. By simply measuring the positions and orientations of trackers attached to femur and tibia and collecting points on bone surfaces, the computer of the navigation system can calculate the shorter distance between the tibial and femoral surfaces, the tibial surface being usually a flat cut.

During prosthesis implantation, it is desired to obtain a good balancing of the joint so that the joint is not too much tightened and not too much loosened. The following explanations will be given regarding knee arthroplasty, but are also true for hip or shoulder arthroplasty. A good balancing is important for obtaining a correct functional knee with maximal range of motion and no extra laxities that generate wear. This process is usually left to the visual and tactile appreciation of the surgeon. In some cases, surgeons use a tensor to impose a given arbitrary pressure between the tibia and the femur in order to determine the optimal relationship between the femur and the tibia at a given flexion angle, and that relationship determines the relative position of the tibial and femoral prosthesis by using mechanical jigs or navigation systems. But a major issue is to determine which pressure should be applied on both compartments, in both flexion and extension, and at various flexion angles. There is no precise standard and no proof that one pressure value is better than another. Sometimes a value of 300 kPa is considered as good, but values of 200 kPa or 400 kPa are also acceptable. In addition, the specific set up of the tensor, the multiple parameters and variables specific to the patient such as weight and its installation generate noise and artifacts that make results not reproducible. For instance, once a tensor is positioned inside the joint at a given pressure, a small variation of the tensor position with a constant pressure can change significantly the relationship between the femur and the tibia. These problems limit considerably the use of a tensor to optimize knee balancing.

It is thus sought a device and a method to determine the patient-specific pressure that needs to be applied during the ligament balancing steps.

### BRIEF DESCRIPTION OF THE INVENTION

A first object of the invention is a computer assisted surgical navigation method for determining the patient-specific pressure that must be applied on one or both sides of a joint during the ligament balancing steps of an arthroplasty procedure, wherein a tensor has previously been inserted inside the joint between a first bone and a second bone when the joint is in a given angular orientation, said method comprising:
controlling the pressure applied by the tensor to the joint so as to make it increase from a minimum value (e.g. zero) to a maximum value;
measuring the evolution of the distance between the first and the second bones during said pressure increase;
determining the relationship between said distance and said pressure;
processing said relationship to determine said patient-specific pressure to apply to the joint.

According to an advantageous embodiment, the method further comprises plotting a curve showing the relationship between the distance and the pressure, thereby displaying the elastic phase and the rigid phase of the ligament, wherein the patient-specific pressure is determined as the pressure that corresponds to the transition between the elastic phase and the rigid phase.

According to a first embodiment of the invention, the pressure is increased continuously at a given speed.

According to another embodiment of the invention, the pressure is applied step by step with a given delay between successive steps.

In the case where the joint is a knee, said given angular orientation of the joint is comprised between 30 and 60°, which corresponds to the range where the balance of the knee is the most relevant to restore the knee function.

A second object of the invention is a surgical device for determining the patient-specific pressure that must be applied on one or both sides of a joint during the ligament balancing steps of an arthroplasty procedure, said device comprising:
a navigation system adapted to track the positions of a first bone and a second bone of a joint;
a tensor adapted to be inserted inside the joint;
a control unit for controlling the pressure applied by said tensor to the joint;
measurement means for measuring the distance between the first bone and the second bone when the pressure applied by the tensor varies;
computing means for determining the relationship between said distance and said pressure;
processing means for determining, based on said relationship, the patient-specific pressure.

According to an advantageous embodiment, the device further comprises plotting means for plotting a curve showing the relationship between the distance and the pressure.

A third object of the invention is the use, in a computer assisted navigational arthroplasty procedure, of:
a tensor connected to a control unit for controlling the pressure applied by the tensor to the joint;
measurement means for measuring the distance between the first bone and the second bone when the pressure applied by the tensor varies;
computing means for determining the relationship between said distance and said pressure;
processing means for determining, based on said relationship, the patient-specific pressure.

### BRIEF DESCRIPTION OF THE DRAWINGS

FIG. 1 is a sequential view showing the device according to the invention.

FIG. 2 shows a frontal view of the femur and tibia with the four tensor plates inserted between the femoral distal condyles and the tibial cut.

FIG. 3 shows an axial view of the femur and tibia with the four tensor plates inserted between the femoral posterior condyles and the tibial cut.

FIG. 4 is a graphical curve showing the behavior of the gap distance that is a function of the pressure applied by the tensor.

FIG. 5 is a surgical procedure flow diagram showing how the user is supposed to interact with the system to calibrate the tensor and then use it for ligament balancing.

### DETAILED DESCRIPTION OF THE INVENTION

For clarity purpose, the description is directed to knee arthroplasty in which a tensor is used on both medial and lateral condyles at one or several flexion angles in order to balance the knee ligaments and obtain good knee stability after prosthesis implantation. It thus concerns the femur as a first bone and the tibia as a second bone.

However, the method can be applied also to other joint replacement procedures such as hip arthroplasty and shoulder arthroplasty.

### Tensor

The Tensor is any surgical instrument with the following characteristics:
The Tensor is a device that is used intraoperatively to control ligament tension at different flexion angles. In most knee surgical procedures, the main active ligaments are the lateral collateral ligament (LCL) and the medial collateral ligament (MCL).
The Tensor delivers several pressures that can be controlled. Two independent pressures are applied, measured and controlled for the knee application. The pressure applied on the medial condyle is independent of the one applied on the lateral condyle.
The technology that is used to apply pressure is independent of the invention. The Tensor mechanism could be active by springs, pneumatic, mechanical, or hydraulic actuators, or passive by manual motion. In all cases, the pressures measured in the Tensor are output.
The pressures are given as output from the Tensor to a computer which is part of the complete system. There are several possibilities:
   i. The Tensor is active and connected to a device that measures the distance between plates. Such device is a standard distance measurement sensor connected to the computer, it can use several technologies such as optical encoders or potentiometers for instance, or it can compute a distance from another physical value such as pressure or volume or a deformable balloon. The communication protocol can be wired such as USB, or wireless, such as radiofrequency, Bluetooth or Wifi. The pressure is given as an input to the Tensor by the computer.
   ii. The Tensor is active but it has no distance measurement inside. The pressure follows a pattern programmed in the computer. In that case, the distance measurement between the two plates is obtained by a navigation system.
   iii. The Tensor is passive. The operator applies some pressures manually and the computer can measure the applied pressures. The distance is obtained by a distance sensor like in (i) or by a navigation system like in (ii).
In all cases, the tensor can be used to vary the pressure on both medial and lateral compartments and measure the corresponding distance between the two plates on each side, defined as “gap distance”.

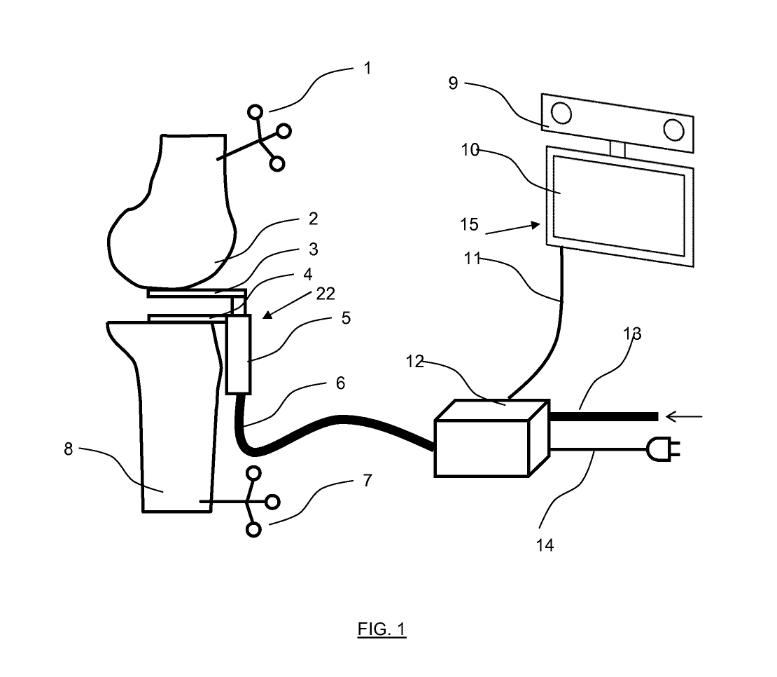

FIG. 1 is a sequential view showing the device according to the invention. It comprises a tensor 22 which is inserted between the tibia 8 and the femur 2, and that is connected to the navigation system 15 through a control unit 12.

The navigation system comprises a camera 9 and a computer with a display 10. These features are well-known from the one skilled in the art and will not be described in detail.

For the rest of this document, and for the explanation of the Invention, the Tensor is active and described as a pneumatic device, activated by air pressure, and the distance measurements are obtained by a navigation system. In that configuration, the computer of the tensor and the computer of the navigation system are merged in one computer.

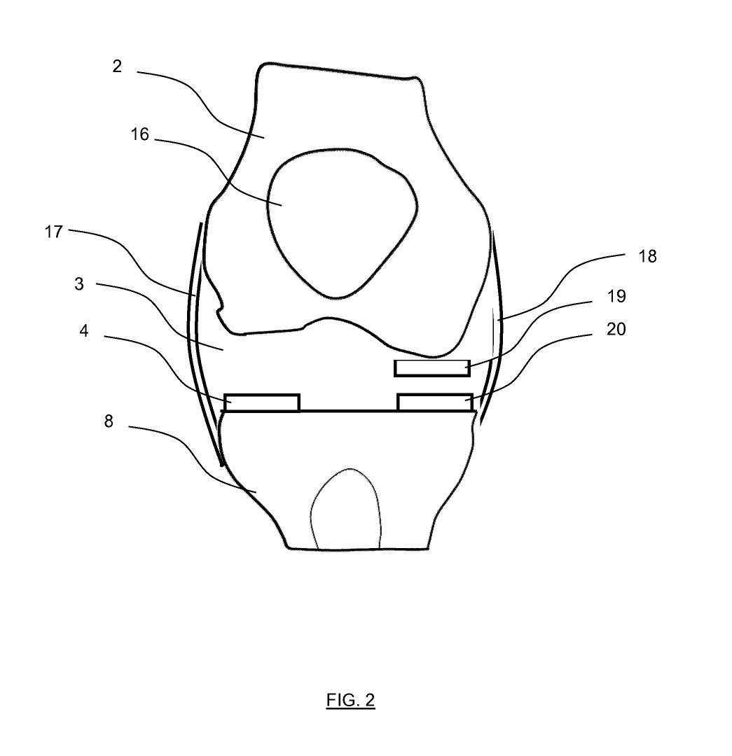

FIG. 2 shows a frontal view of the femur 2, the patella 16, and the tibia 8 with the four tensor plates 3, 4, 19, 20 inserted between the femoral condyles and the tibial cut. On the medial side, one plate 20 is in contact with the tibial cut, and one plate 19 is in contact with the femoral medial condyle. On the lateral side, one plate 4 is in contact with the tibial cut, and one plate 3 is in contact with the femoral lateral condyle. The lateral collateral ligament (LCL) and the medial collateral ligament (MCL) are respectively illustrated as reference numerals 17 and 18. When the pressure increases (resp. decreases) on the medial side, the two plates 19 and 20 are moving apart (resp. closer). When the pressure increases (resp. decreases) on the lateral side, the two plates 3 and 4 are moving apart (resp. closer).

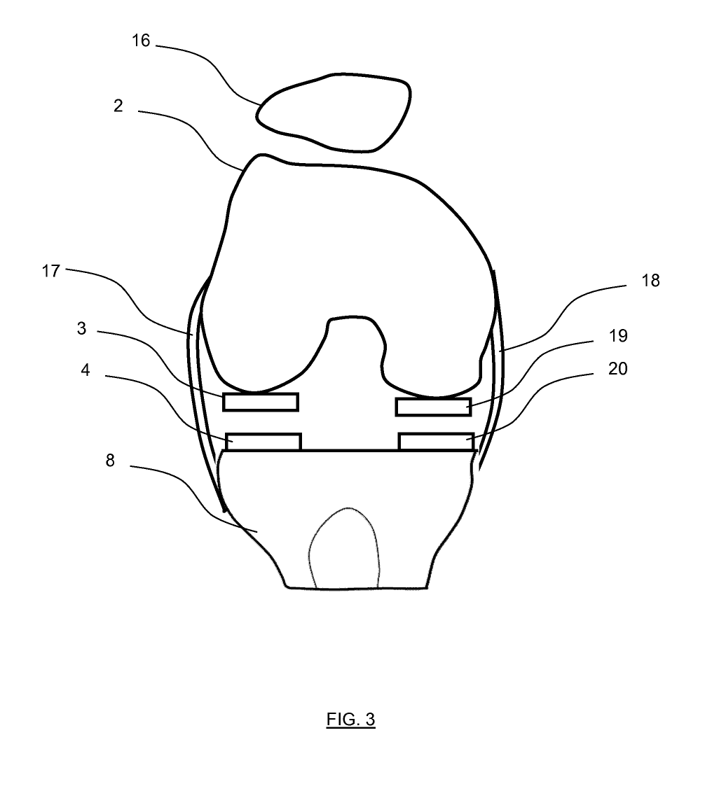

FIG. 3 shows an axial view of the bones and the tensor of FIG. 2.

In one preferred embodiment, the tensor device is an active pneumatic device with a locking mechanism on each plate. When the computer of the system sends a signal, a given plate of the Tensor is immediately locked rigidly to its current position. In that position, the tensor imposes a given gap distance instead of a given pressure. The locking mechanism is for instance an electromagnetic brake applied to the translation of the tensor plate; it can be also a piezoelectric brake, or the like; it can also a manual locking device activated by the surgeon when the computer sends an instruction. There are many possible modes of use of such a locking mechanism used in conjunction with a pneumatic tensor and measurements pressure.

### Principle of Operation

As shown on FIG. 1, a femoral tracker 1 is rigidly fixed on the patient's femur 2, so that the navigation system 15 can track in real-time the position of the femur.

A tibial tracker 7 is rigidly fixed on the patient's tibia 8, so that the navigation system 15 can track in real-time the position of the tibia.

The Control Unit 12 of the Tensor is connected to the air pressure 13 that is available in every standard operating room, powered by the power cable 14, and connected to the navigation system through a USB cable 11.

When the air pressure controlled by the Control Unit 12 is injected into the cable 6, the jacks 5 spread the lower plate 4 from the upper plate 3.

### Automatic Determination of Optimal Pressure

In order to determine which pressure values of the tensor should be used for optimal knee balancing, we propose to use the following method. The joint is at a given flexion angle, for instance in extension, and the tensor is inserted inside the joint. Usually a tibial cut has been performed first in order to insert the tensor. For one or two sides of the knee, internal and external, the pressure is increased progressively from 0 up to a maximal value MP, which is fixed arbitrarily for a given patient. MP is for instance set to 500 kPa for a robust patient and 400 kPa for an elderly and fragile patient. During that process, the distance between the two plates of the tensor between the tibia and the femur, is increasing progressively on both sides of the knee.
In one preferred embodiment, the pressure increases continuously from 0 with a given speed.
In another preferred embodiment, the pressure in increased step by step with a delay between each step.

The speed or the delay depends on the time necessary to stabilize the pressure and the gap distance. This time depends on both the tensor design and the ligament characteristics. When using a pneumatic tensor, it is recommended to vary the pressure slowly and step by step so that the input command in pressure has enough time to establish the real pressure in output equal to the input. But it is also possible to send a command input of a high pressure right away and to observe the pressure curve growing within less than 10 seconds and to measure both pressure output and gap distance simultaneously during that fast variation.

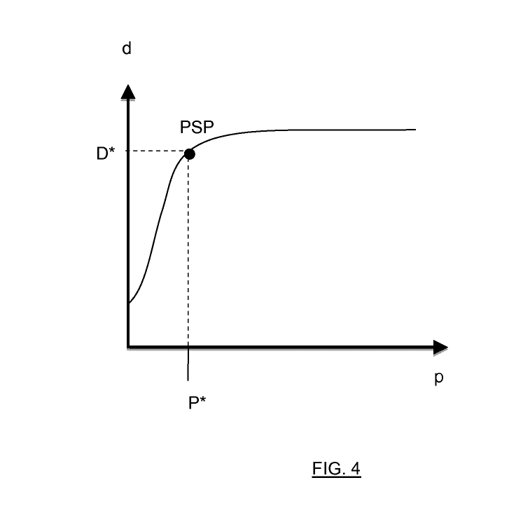

The distances between the contact points between the bones and the plates are measured in real-time. This is trivial if a dedicated distance sensor is used. Alternatively, a navigation system can be used. Such navigation system measures the coordinates of the tibial bone surface, which is usually a planar surface resulting from a tibial cut, in the tibial tracker reference. The navigation system also measures the coordinates of the femoral condyles in the femoral tracker reference. Then, the navigation system can compute in real-time the gap distance d between the tibial cut and the femoral condyle surface, while the pressure p is increased. Therefore, the navigation does not measure directly the gap distance, but indirectly the distance between the femoral condyle surface and the tibial surface. The resulting function F is d=F(p). It is computed and displayed as a curve illustrated in FIG. 4. On this curve, the abscissa represents the pressure p applied by the tensor and the ordinate represents the gap distance d between the tibial cut and the femoral condyle surface.

The Patient-Specific Pressure (PSP) is determined from the pressure curve F described above.
In one preferred embodiment, the PSP is determined automatically as the pressure that separates the elastic phase and the rigid phase of the ligament, as it is illustrated in FIG. 4. When one starts increasing the pressure from 0, the ligament is elastic, which means that the length of the ligament increases proportionally while the pressure increases. Once the PSP has been reached, the ligament enters into a rigid phase, which means that despite the force that is continuously increased, the ligament length does not increase or few. In that phase, the length curve comes to a plateau or to a low slope. Detecting a plateau or a low slope on a curve is done by signal processing, for instance by applying first a low pass filter to remove noise on the curve and then by measuring the variations of the curve in height and comparing it with a threshold. When the local variation or slope of the curve is below a threshold the point is considered to be the PSP. There are many sophisticated algorithms that can be used to detect this transition point. Optionally, it is also possible to add positive or negative small offsets to the plateau point. A negative offset means the final PSP is more conservative with respect to the plateau point. A positive offset means the PSP is more aggressive. As soon as the plateau point is detected, the process can stop and it is not necessary to reach the maximal value MP. The PSP point corresponds to a value of pressure P* and to a gap distance D*.
In another preferred embodiment, the PSP can be adjusted manually by taking into account clinical and patient-specific parameters such as patient age, patient activity, patient size and weight, osteoarthritis stage, preoperative varus/valgus angle. In that method, the pressure curve is displayed progressively, starting from zero and the pressure is slowly increased. The surgeon decides to stop the elongation process before the maximal value MP has been reached. From the portion of pressure curve that has been obtained, the surgeon decides which value PSP is the most appropriate. For instance, in some specific cases, the elastic part can go very far before a plateau can be reached and the surgeon can visually assess this phenomenon and decide that the variation of length has decreased enough to freeze the PSP to the current value. In pathological cases, a variety of patterns of the curve F can be obtained and only an experienced surgeon can decide which value PSP must be used, based on the curve displayed on the screen.

The PSP determined with the previous method can be used in many ways.
In one preferred embodiment, the PSP is calculated for one flexion angle and for one side of the knee. It is then used as a reference value for all other flexion angles and also for the other side of the joint. The PSP pressure is applied only on one side, usually the medial side which corresponds to the most representative ligaments since it is usually less affected by the noise coming from active external structures such as muscles that link the two bones and are partially inactivated by anesthesia. In that case, a different pressure can be applied to the other side of the knee, for instance the tensor pressure is increased or decreased until the leg axis between the Hip Center, the Knee center and the Ankle center, which is the HKA angle, measured continuously by the navigation system reaches a desired value which is usually 180°, whilst the Patient Specific Pressure remains continuously applied to the internal side. For that configuration, the plates can be locked by the locking mechanism and the surgeon can check by applying stress varus and stress valgus manually that it provides a correct sensation of knee stability in extension. Then the knee is placed in flexion, and the previously determined PSP is applied on the internal side or a new PSP is determined with the same method. The pressure on the lateral side is then increased or decreased until the rotation between the tibia plateau and the femur reaches a given value. The plates can be locked in that position. With the help of the computer and appropriate graphical user interface, the surgeon can then plan the thickness of the tibial plateau and the implant size that best matches the desired positions in extension and flexion. The surgeon can also decide to warrant that an extra gap of at least 2 mm exists in flexion for the planned implants with respect to the previously determined positions such that this extra laxity offers enough range of motion of the knee.
In another preferred embodiment, the PSP is calculated for several flexion angles and for both sides independently. For instance, for a given patient, PSP values are determined for continuous angles of flexion, or for two discrete series such as 0°, 10°, 20°, 30° that simulate patient walk and 90°, 100°, 110°, 120° that simulate patient climbing stairs. For each angle, the PSP is determined and the relative position of the tibia and the femur is recorded for the determined PSP. The result is a list of relative positions that indicates an optimal stability of the knee. The graphical user interface of the navigation system can then be used to match the implant size and positions with this pattern. This method can be used only for the internal side and the external side is adjusted in parallel to match standard geometrical features such as HKA equal to 180° in extension and rotation equal to a pre-defined value of 3° at 90° of flexion for instance with all intermediate values being interpolated from those nominal values.

### Surgical Procedure Flow Diagram

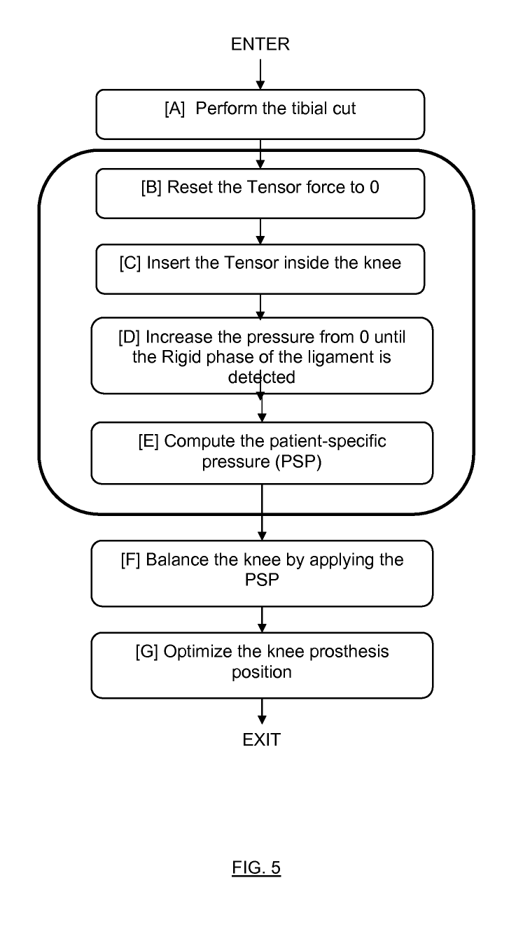

The Invention can be used in the method described in the following Surgical Procedure Flow Diagram of FIG. 5, from step [B] to [E]. The other steps are described for the understanding of the way the method is inserted into an existing computer assisted surgical protocol.
[A] Tibial Cut. The surgeon performs the tibial cut before any use of the Tensor. The cut plane coordinate in the tibial tracker reference is recorded by the navigation system. It can be recorded by tracking in real-time the tibial cutting block, or by digitizing the realized cut after the cut is done.
[B] Tensor Reset. The tensor force is reset to 0, so that it does not create any pressure on the ligaments.
[C] Tensor Insertion. The Tensor is inserted inside the Knee. The space between the tibial cut and the femoral condyle must be large enough to insert the tensor plates. Therefore, the ligaments are not tightened.
[D] Pressure Increase. The pressure is increased progressively from 0, which progressively increases the distance between the tibial cut and the given femoral condyle where the pressure is applied. The navigation system computes the distance between femoral condyles and the tibial cut with respect to the pressure. An elongation versus pressure curve is computed and displayed, said “Pressure Curve”. Details are provided in the section “Automatic Calibration Algorithm”.
[E] Patient-Specific Pressure (PSF) computation. The Patient-Specific Pressure (PSP) is manually selected or automatically computed from the Pressure Curve.
[F] Ligament Balancing. Once the PSP is determined, the surgeon can perform the ligament balancing steps. The surgeon inserts the tensor inside the knee in flexion. The PSP is applied on both medial and lateral posterior condyles. Then the surgeon inserts the tensor inside the knee in extension. The PSP is then applied on both medial and lateral distal condyles. Starting from the extension angle instead of the flexion is also possible.
[G] Knee prosthesis position optimization. The different parameters of the prosthetic components can be adjusted by taking into account the ligament balancing steps. Those parameters include but are not limited to:
   i. Femoral component type, size, position and orientation.
   ii. Tibial component type, size, position and orientation.
   iii. Insert type, size, position and orientation.

### Advantages of the Invention

The conventional techniques that use a tensor to perform the ligament balancing steps during a joint replacement procedure let the surgeon decide which pressure or force need to be applied. The main advantage of the Invention is to propose a device and a method to determine the patient-specific pressure that needs to be applied during the ligament balancing steps.

The second advantage of the Invention is to propose a method which is automated and as a consequence extremely efficient and fast during the surgery.

1. A computer assisted surgical navigation method for determining a patient-specific pressure to be applied on one or both sides of a joint during ligament balancing steps of an arthroplasty procedure, wherein a tensor has previously been inserted inside the joint between a first bone and a second bone when the joint is in a given angular orientation, said method comprising: controlling the pressure applied by the tensor to the joint so as to make said pressure increase from a minimum value to a maximum value; measuring the evolution of the distance between the first and the second bones during said pressure increase; determining the relationship between said distance and said pressure; processing said relationship to determine said patient-specific pressure to apply to the joint.

2. The method of claim 1, further comprising plotting a curve showing a relationship between the distance and the pressure, thereby displaying an elastic phase and a rigid phase of the ligament, wherein the patient-specific pressure is determined as the pressure that correspond to a transition between the elastic phase and the rigid phase.

3. The method of claim 1, wherein the pressure is increased continuously at a given speed.

4. The method of claim 1, wherein the pressure is applied step by step with a given delay between successive steps.

5. The method of claim 1, wherein the joint is a knee and wherein said given angular orientation of the joint is comprised from 30 to 60°.

6. A surgical device for determining a patient-specific pressure to be applied on one or both sides of a joint during ligament balancing steps of an arthroplasty procedure, said device comprising: a navigation system adapted to track positions of a first bone and a second bone of a joint; a tensor adapted to be inserted inside the joint; a control unit for controlling the pressure applied by said tensor to the joint; measurement means for measuring the distance between the first bone and the second bone when the pressure applied by the tensor varies; computing means for determining a relationship between said distance and said pressure; processing means for determining, based on said relationship, the patient-specific pressure.

7. The device of claim 6, further comprising plotting means for plotting a curve showing the relationship between the distance and the pressure.
